(19) United States
(12) Patent Application Publication
(10) Pub. No.: US 2020/0170751 A1
(43) Pub. Date: Jun. 4, 2020

(54) SYSTEM AND METHOD FOR FIDUCIAL ATTACHMENT FOR ORTHOPEDIC SURGICAL PROCEDURES

(71) Applicants: Timothy Pack, Fremont, CA (US); Joel Zuhars, Fremont, CA (US)

(72) Inventors: Timothy Pack, Fremont, CA (US); Joel Zuhars, Fremont, CA (US)

(73) Assignee: THINK SURGICAL, INC., Fremont, CA (US)

(21) Appl. No.: 16/697,919

(22) Filed: Nov. 27, 2019

Related U.S. Application Data
(60) Provisional application No. 62/773,614, filed on Nov. 30, 2018.

(51) Int. Cl. A61B 90/00; A61B 34/20

(52) U.S. Cl. CPC A61B 90/39; A61B 34/20; A61B 2090/363; A61B 2090/3983; A61B 2090/3916; A61B 2034/2055

(57) ABSTRACT

A fiducial attachment device for securement of one or more fiducials to a bone is provided that includes a bracket body having a first hole adapted to receive a first bone pin at a non-parallel angle α and having a second hole adapted to receive a second bone pin at a non-parallel angles α′, the angles α and α′ being relative to a center line of said bracket body between the first hole and the second hole. The bracket body sometimes having an aperture therein for knob attachment. A fiducial attachment member extends away from the bracket body, where a distal end of the fiducial attachment member is an attachment point for the one or more fiducials. A method of using the fiducial attachment device is also provided. A system for using a fiducial attachment device of during a surgical procedure is also provided.

### RELATED APPLICATIONS

This application is a non-provisional application that claims priority benefit of U.S. Provisional Application Ser. No. 62/773,614 filed Nov. 30, 2018, the contents of which are hereby incorporated by reference.

### TECHNICAL FIELD

The present invention generally relates to the field of computer-assisted orthopedic surgery, and in particular to a fiducial attachment device for attaching one or more fiducial markers to a bone.

### BACKGROUND

Throughout a lifetime, bones and joints become damaged and worn through normal use, disease, traumatic events, or a combination thereof. Arthritis is a leading cause of joint damage that leads to cartilage degradation, pain, swelling, stiffness, and bone loss overtime. Arthritis can also cause the muscles articulating the joints to lose strength.

If the pain associated with the dysfunctional joint is not alleviated by less-invasive therapies, a joint arthroplasty procedure is considered as a treatment. Joint arthroplasty is an orthopedic procedure in which an arthritic or dysfunctional joint surface is replaced with an orthopedic prosthesis.

The accurate placement and alignment of an implant is a large factor in determining the success of joint arthroplasty. Even a slight misalignment of the implant may result in poor wear characteristics, reduced functionality, decreased prosthetic longevity, or a combination thereof. Therefore, computer assisted surgical devices are gaining popularity as a tool to pre-operatively plan and precisely execute the plan to ensure an accurate final position and orientation of the prosthetics within the patient's bone that can improve long term clinical outcomes and increase the operational lifespan of the prosthesis. In general, the computer assisted surgical systems include two components, an interactive pre-operative planning software program and a computer assisted surgical device that utilizes the pre-operative data from the software to assist the surgeon in precisely executing the procedure.

Conventional interactive pre-operative planning software generates a three dimensional (3-D) model of the patient's bony anatomy from a computed tomography (CT) or magnetic resonance imaging (MRI) image dataset of the patient. A set of 3-D computer aided design (CAD) models of the manufacturer's prosthesis are pre-loaded in the software that allows the user to place the components of a desired prosthesis to the 3-D model of the boney anatomy to designate the best fit, position and orientation of the implant to the bone. The user can then save this pre-operative planning data to an electronic medium that is loaded and read by a surgical device to assist the surgeon intra-operatively in executing the plan.

In order to achieve accurate implant placement and alignment, a cutting tool (e.g., a saw, drill, end-mill, reamer) is accurately positioned relative to the bone prior to making any bone cuts and/or modifications. In robotic surgical procedures, the cuts are made using a computer-assist device (e.g., a surgical robot) that controls a cutting tool. When a computer-assist device is used to make the cuts, the bone's position and orientation (POSE) must be known precisely in three-dimensional space relative to the computer-assist device to ensure that the cuts and/or modifications are made in the correct location. Several methods to determine the POSE of a bone relative to a computer-assist device are known in the art such as the registration methods described in U.S. Pat. Nos. 6,033,415 and 5,951,475.

After the bone is registered, the position and orientation (POSE) of the bone needs to be monitored or tracked in real-time to ensure the POSE of the bone does not shift relative to the cuts being made with the computer-assisted surgical device. In most computer-assisted surgical procedures, the bone is tracked with an optical tracking system that tracks a tracking array installed and registered to the bone. FIG. 1 illustrates a prior art tracking array 10 shown as installed on a bone B via pins (12a, 12b). The one or more pins (12a, 12b) are inserted straight and perpendicularly into the bone B and an assembly block 14 assembles or attaches the pins (12a, 12b) to the tracking array 10. The tracking array 10 permits the bone B to be tracked by the optical tracking system in real-time.

However, the placement of fiducial bodies onto bones can be problematic and invasive. The tracking array 10 needs to be rigidly fixed to the bone such that there is no movement between the tracking array 10 and the bone B during the procedure. If any relative movement occurs, the cuts made on the bone B will be shifted because the positional relationship previously established between the tracking array 10 and the bone B is no longer valid. Conventional practice has required the use of large diameter screws and pins to ensure a rigid relationship, which are undesirable as these types of screws and pins may increase fracture risk and postoperative pain. Ideally, a fiducial body is easily attached, forms a rigid connection to the bone, and requires minimal removal and displacement of soft tissues.

Thus, there is a need in the art for devices and methods for attaching fiducial markers to overcome the aforementioned problems of the prior art. There further exists a need to provide at least attribute for fiducial marker placement of being easily attached to bone, forming a rigid connection to the bone, or involving minimal removal and displacement of soft tissues.

### SUMMARY OF THE INVENTION

A fiducial attachment device for securement of one or more fiducials to a bone is provided that includes a bracket body having a first hole adapted to receive a first bone pin at a non-parallel angle α and having a second hole adapted to receive a second bone pin at a non-parallel angles α′, the angles α and α′ being relative to a center line of said bracket body between the first hole and the second hole. A fiducial attachment member extends away from the bracket body, where a distal end of the fiducial attachment member is an attachment point for the one or more fiducials.

Another fiducial attachment device for securement of one or more fiducials to a bone is also provided based on a bracket body having a first hole adapted to receive a first bone pin at a non-parallel angle α and having a second hole adapted to receive a second bone pin at a non-parallel angles α′, the angles α and α′ being relative to a center line of said bracket body between the first hole and the second hole, said bracket body having a first aperture therein. A first knob engaging the first aperture and positioned between the first hole and the second hole and adapted to simultaneously engage bone pins in the first hole and the second hole. A fiducial attachment member extending away from the bracket body, where a distal end of the fiducial attachment member is an attachment point for the one or more fiducials.

A method of using the fiducial attachment device includes a pair of pins being screwed into the bone at non-parallel angles with a body bracket. One or more fiducials are then attached to the fiducial attachment member.

A system for using a fiducial attachment device of during a surgical procedure includes a tracking system that generates position and orientation (POSE) data of the bone based on the one or more fiducials attached to the bone with the fiducial attachment device. A robot is provided with a set of tools for the surgical procedure. One or more computers having one or more processors are provided for controlling the robot based on the POSE data with respect to a surgical plan.

### BRIEF DESCRIPTION OF THE DRAWINGS

Examples illustrative of embodiments of the present invention are described below with reference to figures attached hereto. In the figures, identical structures, elements or parts that appear in more than one figure are generally labeled with a same numeral in all the figures in which they appear. Dimensions of components and features shown in the figures are generally chosen for convenience and clarity of presentation and are not necessarily shown to scale. The figures are listed below.

FIG. 1 depicts a prior art tracking array installed in a bone;

FIG. 2 depicts a fiducial attachment device assembled to a bone and a fiducial marker array attached thereof in accordance with embodiments of the invention;

FIGS. 3A and 3B depict the fiducial attachment device of FIG. 2 shown in perspective and side views, respectively;

FIGS. 4A and 4B depict a fiducial attachment device with a set of adjustment knobs shown in perspective and side views, respectively in accordance with embodiments of the invention;

FIG. 5 is an exploded view of the fiducial attachment device with a set of adjustment knobs of FIGS. 4A and 4B in accordance with embodiments of the invention; and

FIG. 6 illustrates a surgical system in the context of an operating room (OR) in accordance with embodiments of the invention.

### DETAILED DESCRIPTION

The present invention has utility as a device and method for aiding a surgical team in installing a set of fiducial markers on a bone. Embodiments of the inventive fiducial attachment device and method use a bracket body to guide the placement of two pins into the bone at slight angles toward one another and with respect to the center line of the bracket body. The two angled pins are then secured by wedging a conic screw or cam between the two pins that applies an outward force to the pins. The secure attachment of embodiments of the inventive fiducial attachment device improves stress distribution on the bone compared to using parallel pins alone as in prior art designs by spreading forces along the contacting surface between the bone and bracket body.

In a specific embodiment of the inventive fiducial attachment device, a bracket is placed requiring only the use of one screw that is perpendicular to the bone contacting surface. The driving of the single screw wedges the bracket body against the bone forming a robust attachment with better stress distribution than pins or screws alone. The robust fixation and attachment of the fiducial attachment device is achieved with less damage (smaller holes) to bone than using multiple screws or large diameter screws common in prior art designs.

The present invention will now be described with reference to the following embodiments. As is apparent by these descriptions, this invention can be embodied in different forms and should not be construed as limited to the embodiments set forth herein. Rather, these embodiments are provided so that this disclosure will be thorough and complete, and will fully convey the scope of the invention to those skilled in the art. For example, features illustrated with respect to one embodiment can be incorporated into other embodiments, and features illustrated with respect to a particular embodiment can be deleted from that embodiment. In addition, numerous variations and additions to the embodiments suggested herein will be apparent to those skilled in the art in light of the instant disclosure, which do not depart from the instant invention. Hence, the following specification is intended to illustrate some particular embodiments of the invention, and not to exhaustively specify all permutations, combinations and variations thereof.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents, and other references mentioned herein are incorporated by reference in their entirety.

### Definitions

Unless indicated otherwise, explicitly or by context, the following terms are used herein as set forth below.

As used in the description of the invention and the appended claims, the singular forms “a,” “an” and “the” are intended to include the plural forms as well, unless the context clearly indicates otherwise.

Also, as used herein, “and/or” refers to and encompasses any and all possible combinations of one or more of the associated listed items, as well as the lack of combinations when interpreted in the alternative (“or”).

As used herein, the term “fiducial marker” refers to a physical reference marker designed to permit a measurement system, such as a mechanical tracking system, optical tracking system, electro-magnetic tracking system, ultrasound tracking system, and/or an imaging system (e.g., computed tomography (CT), X-ray, fluoroscopy, ultrasound, magnetic resonance imaging (MRI)), to determine at least one of a position or orientation of at least a portion of the reference marker.

As used herein, the term “registration” refers to the determination of the POSE and/or coordinate transformation between two or more objects or coordinate systems such as a computer-assist device, a bone, pre-operative bone data, surgical planning data (i.e., an implant model, cut-file, virtual boundaries, virtual planes, cutting parameters associated with or defined relative to the pre-operative bone data), and any external landmarks (e.g., a fiducial marker array) associated with the bone, if such landmarks exist. Methods of registration known in the art are described in U.S. Pat. Nos. 6,033,415, 8,010,177, and 8,287,522. “Re-registration” refers to any subsequent registration procedure that occurs after an initial registration.

As used herein, the term “cut volume” refers to a volume of a bone to be removed by a computer-assist device.

As used herein, the term “digitizer” refers to a measuring device capable of measuring physical coordinates in three-dimensional space. Examples of a “digitizer” include a high-resolution electro-mechanical sensor arm as described in U.S. Pat. No. 6,033,415, an optically tracked probe as described in U.S. Pat. No. 7,043,961, and similar measuring devices that may be tracked by other tracking systems known in the art.

As used herein, the term “digitizing” refers to the collection, recordation, or measurement of one or more physical coordinates in three-dimensional space.

As used herein, the term “real-time” refers to processor in which input data is processed within milliseconds such that calculated values are available within 10 seconds of computational initiation.

Also, referenced herein are computer-assist devices, which may also be referred to as computer-assisted devices, computer-assisted surgical systems, and robotic surgical systems. Examples of computer-assist devices illustratively include a 1-N degree of freedom hand-held surgical system, an optical tracking system tracking one or more tools (e.g., tracked instruments, manipulator arms) in space, a navigated surgical system, a serial-chain manipulator system, a parallel robotic system, or a master-slave robotic system, as described in U.S. Pat. Nos. 5,086,401, 6,033,415, 7,206,626, 8,876,830 and 8,961,536, U.S. Pat. App. No. 2013/0060278, 2005/0216032 and U.S. Prov. App. No. 62/054,009 all of which are incorporated by reference herein in their entirety. The computer-assisted surgical system may provide autonomous, semi-autonomous, haptic, or no (passive) control, or any combination thereof.

It is to be understood that in instances where a range of values are provided that the range is intended to encompass not only the end point values of the range but also intermediate values of the range as explicitly being included within the range and varying by the last significant figure of the range. By way of example, a recited range of from 1 to 4 is intended to include 1-2, 1-3, 2-4, 3-4, and 1-4.

While the present invention is illustrated visually hereafter with respect to a femur as the bone for which embodiments of the inventive fiducial attachment device are installed and to which the present invention is applied, it is appreciated that the present invention is equally applicable to other bones of a human, non-human primate, or other mammals.

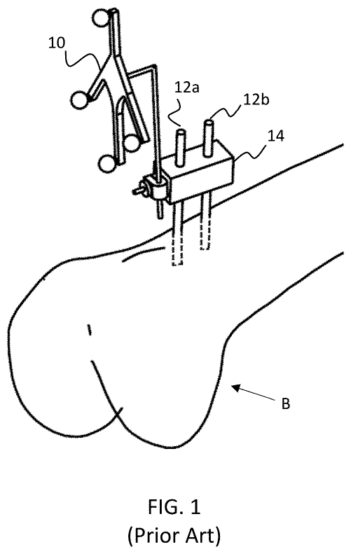

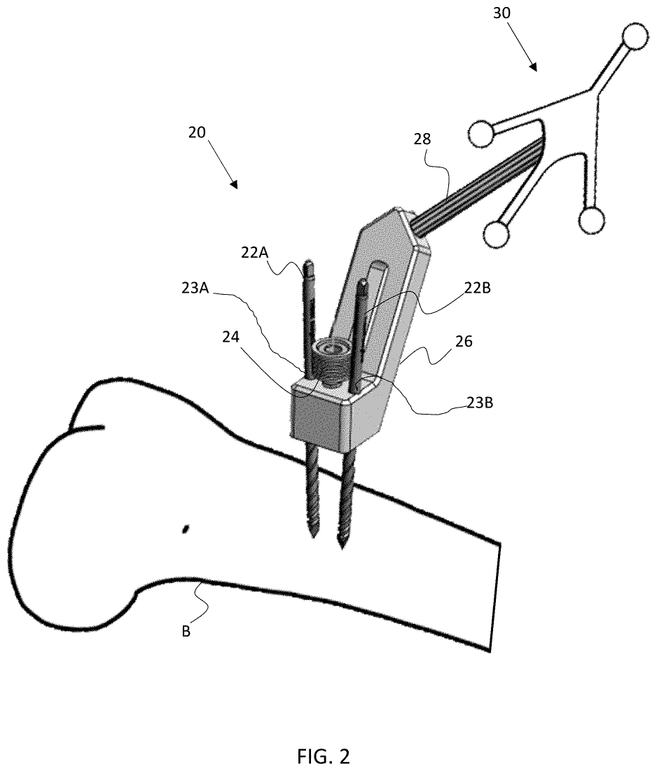

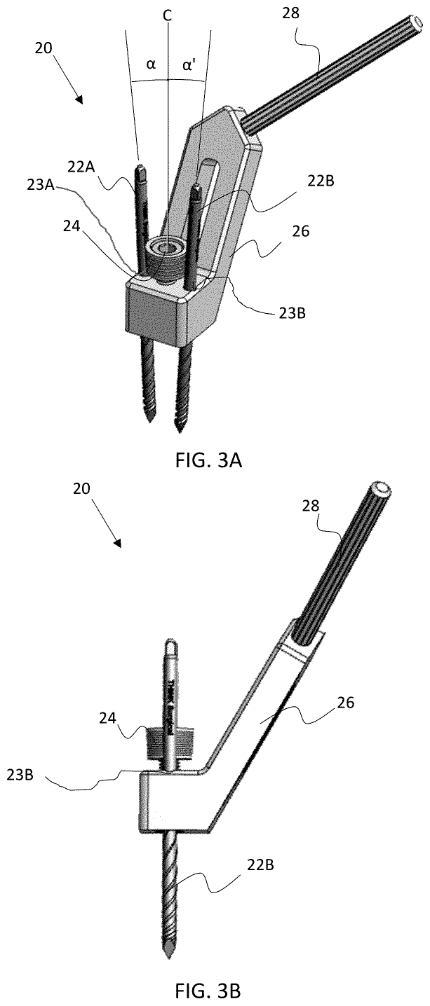

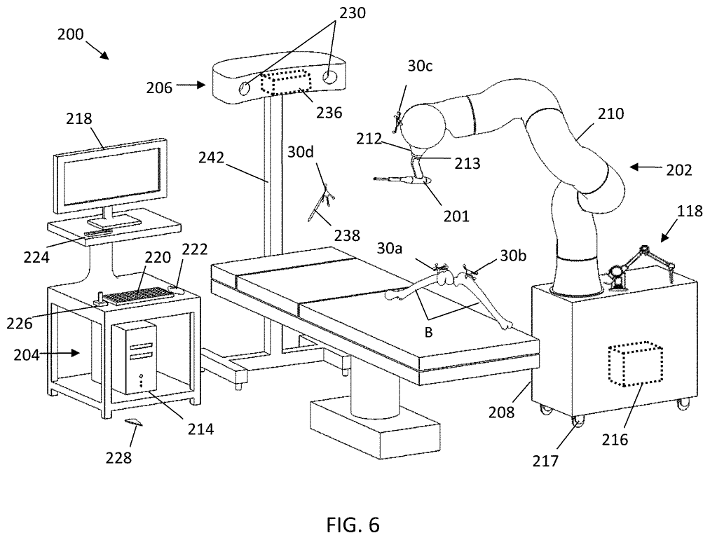

With reference to the figures, FIG. 2 illustrates an inventive embodiment of a fiducial attachment device 20 assembled to a bone B. The fiducial attachment device 20 supports a fiducial marker array 30 attached at a distal end of a fiducial attachment member 28 in the form of rod, bar, or screw that extends upward from a bracket body 26 of the fiducial attachment device 20. The fiducial marker array 30 provides a position and orientation reference based on a variety of techniques illustratively including visual, laser ranging, radio frequency, infrared, or acoustic detection as detailed below with respect to FIG. 6. A set of bone pins (22A, 22B) extend downward from the bracket body 26 for insertion into the bone B through holes 23A and 23B. As best shown in FIG. 3A, the bone pins (22A, 22B) are held at angle alpha (α) and α′, respectively with respect to a centerline (C) of the bracket body 26. The angles α, and α′ in specific embodiments vary independently between 0.5 and 30 degrees relative to C. While in some inventive embodiments, the angles α and α′ are within 5% of one another, in some inventive embodiments, the angles α and α′ are between 5 and 60% of one another with asymmetric angles being particularly advantageous for placement adjacent to rapidly changing bone topography or bone defects. The bracket body 26 guides the placement of the two bone pins (22A, 22B) into bone at the angles α toward one another. The two angled bone pins (22A, 22B) are then secured by wedging a head of a conic screw 24 between the two bone pins (22A, 22B). The secure attachment of embodiments of the inventive fiducial attachment device 20 improves stress distribution on the bone B compared to using parallel pins alone as in prior art designs (FIG. 1) by spreading forces along the contacting surface between the bone B and bracket body 26. Cantilever forces from the two bone pins (22A, 22B) are distributed to the outer edges of the bracket body 26.

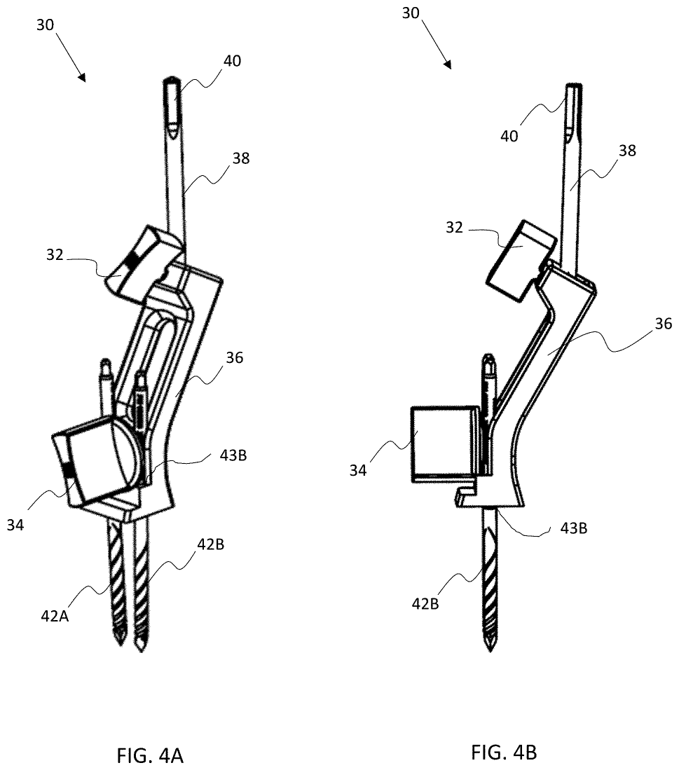

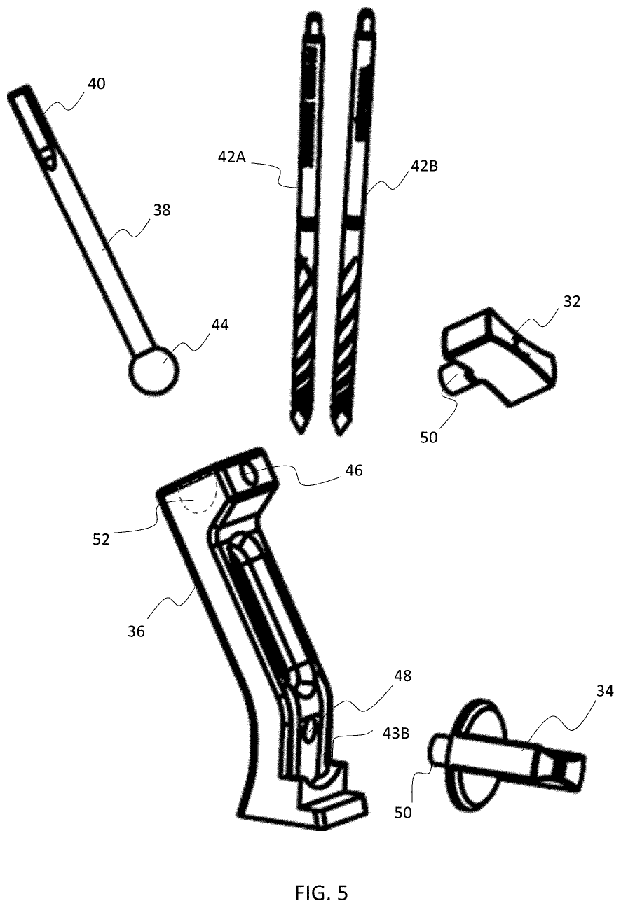

FIGS. 4A and 4B depict an embodiment of an inventive fiducial attachment device 30 with a set of adjustment handles referred to as torque knob A and torque knob B (32, 34, respectively). The fiducial attachment device 30 supports a fiducial marker array that may be attached at a distal end 40 of a fiducial attachment member 38. The fiducial attachment member 38 in the form of rod or bar extends upward from a bracket body 36 of the fiducial attachment device 30. As shown in FIG. 5, the proximal end of the fiducial attachment member 38 has a ball joint 44 that mates with a socket 52 in the bracket body 36. The ball joint 44 and socket 52 combination allows for adjustment of the orientation of the fiducial attachment member 38. Torque knob A 32 screws into aperture 46 in the bracket body 36 and engages and locks the ball joint 44 in the socket 52. A set of bone pins (42A, 42B) extend downward from the bracket body 36 for insertion into a bone through a pair of holes 43A and 43B, respectively. In a similar manner to FIG. 3A, the bone pins (42A, 42B) are held at an angles alpha (α) and α′ with respect to a centerline (C) of the bracket body 36 as detailed above with respect to bone bines 22A and 22B. The bracket body 36 guides the placement of the two bone pins (42A, 42B) into bone at the angles α and α′ toward one another. The secure attachment of embodiments of the inventive fiducial attachment device 30 improves stress distribution on the bone B compared to using parallel pins alone as in prior art designs (FIG. 1) by spreading forces along the contacting surface between the bone B and bracket body 26. The two angled bone pins (42A, 42B) are then secured by torque knob B 34 positioned between the two bone pins (42A, 42B). As best shown in the exploded view of FIG. 5, torque knob B 34 screws into aperture 48 in the bracket body 36, while a screw/pin 50 wedges against the bone pins (42A, 42B). In a specific inventive embodiment, a screw/pin 50 is in the form of a cam that wedges against the bone pins (42A, 42B). Cantilever forces from the two bone pins (42A, 42B) are distributed to the outer edges of the bracket body 46.

### Computer-Assist Device

With reference to FIG. 6, an embodiment of a computer-assist device, and more specifically a robotic surgical system is shown generally at 200 capable of implementing embodiments of the inventive method described herein is shown in the context of an operating room (OR). The surgical system 200 generally includes a surgical robot 202, a computing system 204, and includes at least one of a mechanical digitizer 118 or a non-mechanical tracking system 206 (e.g., optical tracking system).

The surgical system 200 generally includes a surgical robot 202, a computing system 204, and a tracking system 206.

The surgical robot 202 may include a movable base 208, a manipulator arm 210 connected to the base 208, an end-effector 201 located at a distal end 212 of the manipulator arm 210, and a force sensor 213 positioned proximal to the end-effector 201 for sensing forces experienced on the end-effector 201. The base 208 includes a set of wheels 217 to maneuver the base 208, which may be fixed into position using a braking mechanism such as a hydraulic brake. The base 208 may further include an actuator to adjust the height of the manipulator arm 210. The manipulator arm 210 includes various joints and links to manipulate the end-effector 201 in various degrees of freedom. The joints are illustratively prismatic, revolute, spherical, or a combination thereof.

The computing system 204 generally includes a planning computer 214; a device computer 216; a tracking computer 236; and peripheral devices. The planning computer 214, device computer 216, and tracking computer 236 may be separate entities, one-in-the-same, or combinations thereof depending on the surgical system. Further, in some embodiments, a combination of the planning computer 214, the device computer 216, and/or tracking computer 236 are connected via a wired or wireless communication. The peripheral devices allow a user to interface with the surgical system components and may include: one or more user-interfaces, such as a display or monitor 218 for the graphical user interface (GUI); and user-input mechanisms, such as a keyboard 220, mouse 222, pendent 224, joystick 226, foot pedal 228, or the monitor 218 in some inventive embodiments has touchscreen capabilities.

The planning computer 214 contains hardware (e.g., processors, controllers, and/or memory), software, data and utilities that are in some inventive embodiments dedicated to the planning of a surgical procedure, either pre-operatively or intra-operatively. This may include reading medical imaging data, segmenting imaging data, constructing three-dimensional (3D) virtual models, storing computer-aided design (CAD) files, providing various functions or widgets to aid a user in planning the surgical procedure, and generating surgical plan data. The final surgical plan may include pre-operative bone data, patient data, implant position data, trajectory parameters, and/or operational data. The operational data may be a set of instructions for modifying a volume of tissue that is defined relative to the anatomy, such as a set of cutting parameters (e.g., cut paths, velocities) in a cut-file to autonomously modify the volume of bone, a set of virtual boundaries defined to haptically constrain a tool within the defined boundaries to modify the bone, a set of planes or drill holes to drill pins in the bone, a graphically navigated set of instructions for modifying the tissue, and the trajectory parameters for robotic insertion of an implant. In particular inventive embodiments, the operational data specifically includes a cut-file for execution by a surgical robot to autonomously modify the volume of bone, which is advantageous from an accuracy and usability perspective. The surgical plan data generated from the planning computer 214 may be transferred to the device computer 216 and/or tracking computer 236 through a wired or wireless connection in the operating room (OR); or transferred via a non-transient data storage medium (e.g., a compact disc (CD), a portable universal serial bus (USB) drive) if the planning computer 214 is located outside the OR.

The device computer 216 in some inventive embodiments is housed in the moveable base 208 and contains hardware, software, data and utilities that are preferably dedicated to the operation of the surgical device 202. This may include surgical device control, robotic manipulator control, the processing of kinematic and inverse kinematic data, the execution of registration algorithms, the execution of calibration routines, the execution of operational data (e.g., cut-files, the trajectory parameters), coordinate transformation processing, providing workflow instructions to a user, and utilizing position and orientation (POSE) data from the tracking system 206. In some embodiments, the surgical system 200 includes a mechanical digitizer arm 118 attached to the base 208. The digitizer arm 118 may have its own tracking computer or may be directly connected with the device computer 216.

The tracking system 206 may be an optical tracking system that includes two or more optical receivers 230 to detect the position of fiducial markers (e.g., retroreflective spheres, active light emitting diodes (LEDs)) uniquely arranged on rigid bodies. The fiducial markers arranged on a rigid body are collectively referred to as a tracking array (30a, 30b, 30c, 30d) as described above. It should be appreciated, that the rigid body may be part of a surgical device (e.g., manipulator arm 210, end-effector 201, digitizer 238) itself, where the fiducial markers are directly attached or integrated with the surgical device. An example of an optical tracking system is described in U.S. Pat. No. 6,061,644. The tracking system 206 may be built into a surgical light, located on a boom, a stand 242, or built into the walls or ceilings of the OR. The tracking system computer 236 may include tracking hardware, software, data and utilities to determine the POSE of objects (e.g., bones B, surgical device 202) in a local or global coordinate frame. The POSE of the objects is collectively referred to herein as POSE data, where this POSE data may be communicated to the device computer 216 through a wired or wireless connection. Alternatively, the device computer 216 may determine the POSE data using the position of the fiducial markers detected from the optical receivers 230 directly.

The POSE data is determined using the position data detected from the optical receivers 230 and operations/processes such as image processing, image filtering, triangulation algorithms, geometric relationship processing, registration algorithms, calibration algorithms, and coordinate transformation processing.

The POSE data is used by the computing system 204 during the procedure to update the POSE and/or coordinate transforms of the bone B, the surgical plan, and the surgical robot 202 as the manipulator arm 210 and/or bone B move during the procedure, such that the surgical robot 202 can accurately execute the surgical plan.

### Other Embodiments

Patents and publications detailed herein are representative of the skill in art at the time of the present invention. These references are hereby incorporated by reference to the same extent as if each patent or publication was specifically and individually incorporated by reference. While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the described embodiments in any way. Rather, the foregoing detailed description will provide those skilled in the art with a convenient roadmap for implementing the exemplary embodiment or exemplary embodiments. It should be understood that various changes may be made in the function and arrangement of elements without departing from the scope as set forth in the appended claims and the legal equivalents thereof.

1. A fiducial attachment device for securement of one or more fiducials to a bone, comprising:
a bracket body having a first hole adapted to receive a first bone pin at a non-parallel angle α and having a second hole adapted to receive a second bone pin at a non-parallel angles α′, the angles α and α′ being relative to a center line of said bracket body between the first hole and the second hole; and
a fiducial attachment member extending away from said bracket body, where a distal end of said fiducial attachment member is an attachment point for the one or more fiducials.

2. The device of claim 1 further comprising a conic screw positioned between the first hole and the second hole and adapted to simultaneously engage said bracket body and bone pins in the first hole and the second hole.

3. The device of claim 1 wherein said fiducial attachment member is one of a rod, bar, or screw.

4. The device of claim 1 wherein said angles α and α′ are each independently between 0.5 and 30 degrees.

5. The device of claim 1 wherein said angles α and α′ are within 5% of one another.

6. The device of claim 1 wherein said angles α and α′ are between 5% and 60% of one another.

7. The device of claim 1 further comprising said first bone pin and said second bone pin.

8. A fiducial attachment device for securement of one or more fiducials to a bone, comprising:
a bracket body having a first hole adapted to receive a first bone pin at a non-parallel angle α and having a second hole adapted to receive a second bone pin at a non-parallel angles α′, the angles α and α′ being relative to a center line of said bracket body between the first hole and the second hole, said bracket body having a first aperture therein;
a first knob engaging the first aperture and positioned between the first hole and the second hole and adapted to simultaneously engage bone pins in the first hole and the second hole; and
a fiducial attachment member extending away from said bracket body, where a distal end of said fiducial attachment member is an attachment point for the one or more fiducials.

9. The device of claim 8 further comprising a screw or pin to couple said first knob to the first aperture.

10. The device of claim 9 further comprising a cam in mechanical communication with said screw or pin and the bone pins.

11. The device of claim 8 wherein said fiducial attachment member is one of a rod, bar, or screw having a proximal end that terminates in a ball joint that mates with a socket in said bracket body.

12. The device of claim 11 further comprising a second knob that engages a second aperture in said bracket body that mechanically engages said socket and locks the position of said ball joint.

13. The device of claim 8 wherein said angles α and α′ are each independently between 0.5 and 30 degrees.

14. The device of claim 8 wherein said angles α and α′ are within 5% of one another.

15. The device of claim 8 wherein said angles α and α′ are between 5% and 60% of one another.

16. The device of claim 8 further comprising said first bone pin and said second bone pin.

17. A method of using the fiducial attachment device comprising:
screwing a pair of pins into the bone at non-parallel angles with a body bracket; and
attaching the one or more fiducials to a fiducial attachment member.

18. A system for using the fiducial attachment device of claim 1 during a surgical procedure, said system comprising:
a tracking system that generates position and orientation (POSE) data of the bone based on the one or more fiducials attached to the bone with said fiducial attachment device;
a robot with a set of tools for the surgical procedure; and
one or more computers having one or more processors for controlling the robot based on the POSE data with respect to a surgical plan.
